(19) United States
(12) Patent Application Publication
(10) Pub. No.: US 2026/0325810 A1
(43) Pub. Date: Oct. 22, 2026

(54) BODY-INSERTABLE MICRONEEDLE MEDICAL DEVICE

(71) Applicant: SOLIS MSN CO.,LTD, Cheonan-si Chungcheongnam-do (KR)

(72) Inventor: Kang Mo RYU, Cheonan-si Chungcheongnam-do (KR)

(21) Appl. No.: 19/463,166

(22) Filed: Jan. 29, 2026

Related U.S. Application Data
(63) Continuation of application No. PCT/KR2024/002224, filed on Feb. 21, 2024.

(30) Foreign Application Priority Data
Aug. 3, 2023 (KR) 10-2023-0101402

(51) Int. Cl. A61M 37/00; A61B 1/303

(52) U.S. Cl. CPC A61M 37/0015; A61B 1/303; A61M 2210/1475

(57) ABSTRACT

Provided is a technology by which a medical device having a microneedle is inserted into the vagina of a woman to perform various procedures through the microneedle and has a structure for preventing cross-infection between people who have received gynecological treatment. The present invention includes: a handle module shaped to be held by the hand of an operator, the handle module having one side connected to a cable to which external power is supplied, including a driving means installed therein, and having a moving shaft of the driving means, protruding from the side opposite the cable; and a body insertion module detachably coupled to one side of the handle module, the body insertion module receiving the driving force from the driving means while inserted into the body of a patient and allowing a plurality of microneedles to penetrate the body of the patient when exposed externally.

### TECHNICAL FIELD

The present invention relates to a body-insertable microneedle medical device, and more particularly, to medical technology designed to perform various procedures through microneedles by inserting a medical device equipped with the microneedles into the vaginal cavity, and to provide a structure configured to prevent cross-infection among patients undergoing gynecological treatment.

### BACKGROUND ART

With the advancement of medical technology, microneedles having diameters and heights ranging from tens to hundreds of micrometers (μm) have been used to treat various skin diseases, wounds, or aging. Compared to injection methods of the related art, such microneedles cause minimal pain and do not cause skin damage. Furthermore, since the microneedles provide excellent drug delivery efficiency, research and development has been actively conducted thereon. Additionally, the effectiveness of medical treatment is further enhanced by electrical stimulation delivered via the microneedles.

Such problems related to skin diseases and aging affect not only the externally visible skin but also the internal areas that are not visible from the outside. Particularly, as women age and experience pregnancy and childbirth, the skin of the vagina located inside the vulva also undergoes changes.

Specifically, the elasticity of the vaginal mucosa decreases and the internal environment of the vagina becomes dry, leading to various problems such as vaginal dryness, vaginal atrophy, vaginal laxity, vaginitis, dyspareunia, and urinary incontinence. Such problems may cause not only physical pain but also a loss of vitality and self-confidence, resulting in emotional distress such as the avoidance of social interaction.

Particularly, since vaginal aging progresses more rapidly than general skin aging, proactive management is required. If management of vaginal aging is neglected or left untreated, various diseases may develop, and marital relationships may be adversely affected.

In the related art, Korean Patent Laid-Open Publication No. 10-2023-0068596 (entitled “HIGH-FREQUENCY NEEDLE DEVICE FOR VAGINAL TREATMENT”) discloses technology including an insertion body inserted into the vagina of a patient, a needle module provided with a plurality of needles protruding from the interior of the insertion body to the exterior thereof so as to penetrate vaginal inner wall tissues, a driving unit configured to move the needle module such that the plurality of needles protrude to the exterior of the insertion body, and a high-frequency generator configured to use each of the plurality of needles as an electrode to transmit high-frequency energy to the vaginal inner wall tissues through the plurality of needles that have penetrated the tissues.

In the related art, since a grip portion gripped by the hand of an operator and a portion inserted into the patient's body are integrally formed, maintenance such as cleaning and sterilization is required after use of the device. Further, there is a high risk of viral and bacterial cross-infection, and the entire device needs to be replaced in the event of malfunction.

### DISCLOSURE

### Technical Problem

Therefore, the present invention has been made in view of the above problems, and it is an object of the present invention to provide a body-insertable microneedle medical device configured such that an insertion portion of the medical device is inserted into the vaginal cavity, microneedles are deployable from the insertion portion to effectively penetrate a vaginal skin layer, and only the insertion portion is detachable and replaceable after a single use to prevent bacterial and viral infections.

### Technical Solution

In accordance with the present invention, the above and other objects can be accomplished by the provision of a body-insertable microneedle medical device including a handle module formed to be gripped by a hand of an operator, wherein the handle module has one end connected to a cable configured to receive external power, includes a driving means installed therein, and is configured such that a moving shaft of the driving means protrudes from a side opposite to the cable, and a body-insertable module detachably coupled to the other end of the handle module, wherein the body-insertable module is configured to receive driving force from the driving means in a state of being inserted into a body of a patient, such that a plurality of microneedles are exposed to an outside of the body-insertable module to penetrate the body of the patient.

The driving means of the handle module is implemented as a linear actuator such that the moving shaft performs linear motion.

The body-insertable module includes a case having a plurality of needle holes formed through an upper portion thereof, guides integrally formed inside the case, a needle block insertably coupled to and guided by the guides and formed to have the plurality of microneedles fixed to an upper portion thereof, and a push bar having one end coupled to the needle block and the other end coupled to the moving shaft to move the needle block by power of the driving means.

The microneedles of the needle block are obliquely fixed in the diagonal direction at a predetermined angle, and the guides are formed to correspond to inclination angles of the microneedles such that the needle block moves upwards along the inclination angles.

The needle block and the push bar are coupled to each other via a link bar having a first rotating part and a second rotating part.

The case may be made of a transparent material, and an imaging means equipped with a camera may be further installed below the push bar to monitor an inside of the body in real time during an insertion process.

### Advantageous Effects

In a body-insertable microneedle medical device according to the present invention, a body-insertable module and a handle module are not integrally formed but are independently configured in a detachable manner. Accordingly, the body-insertable module, which is the portion inserted into the body, is discarded after a single use and is replaced with a new body-insertable module, thereby having an effect of preventing bacterial and viral infections resulting from repeated use of the body-insertable module.

### DESCRIPTION OF DRAWINGS

The above and other objects, features and other advantages of the present invention will be more clearly understood from the following detailed description taken in conjunction with the accompanying drawings, in which:

FIG. 1 is a perspective view of a body-insertable microneedle medical device according to the present invention;

FIG. 2 is a perspective view showing an exploded state of a handle module and a body-insertable module of the body-insertable microneedle medical device according to the present invention;

FIG. 3 is a partial cross-sectional view showing the exploded state of the handle module and the body-insertable module of the body-insertable microneedle medical device according to the present invention;

FIG. 4 is an enlarged cross-sectional view showing an internal structure of the body-insertable microneedle medical device according to the present invention;

FIG. 5 is an exploded perspective view showing a main configuration of the body-insertable module according to the present invention;

FIG. 6 is an exemplary view showing an operation example of the body-insertable microneedle medical device according to the present invention;

FIG. 7 is a plan cross-sectional view showing the main configuration of the body-insertable module according to the present invention; and

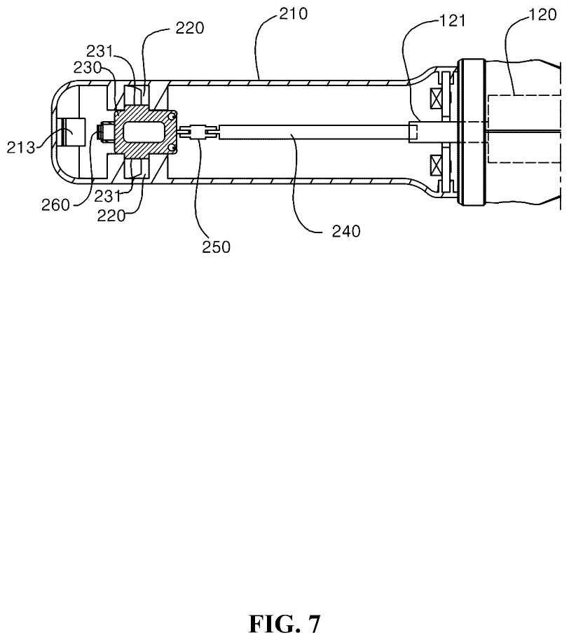

FIG. 8 is a view showing another embodiment of the body-insertable microneedle medical device according to the present invention.

### BEST MODE

Hereinafter, preferred embodiments of the present invention will be described in detail with reference to the attached drawings. Further, in describing the present invention, when it is determined that a detailed description of publicly known functions or configurations may obscure the gist of the present disclosure, detailed description thereof will be omitted.

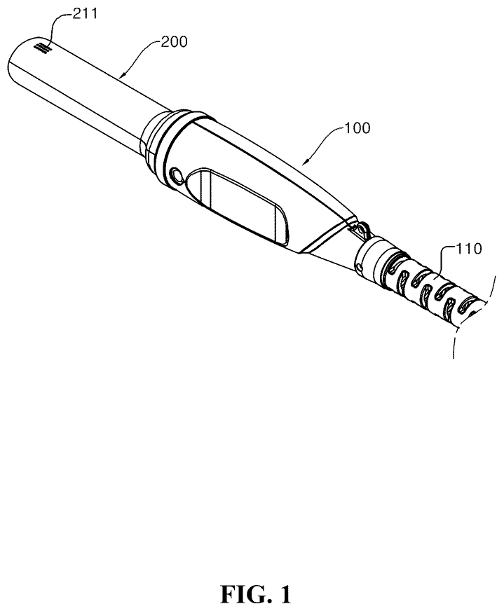

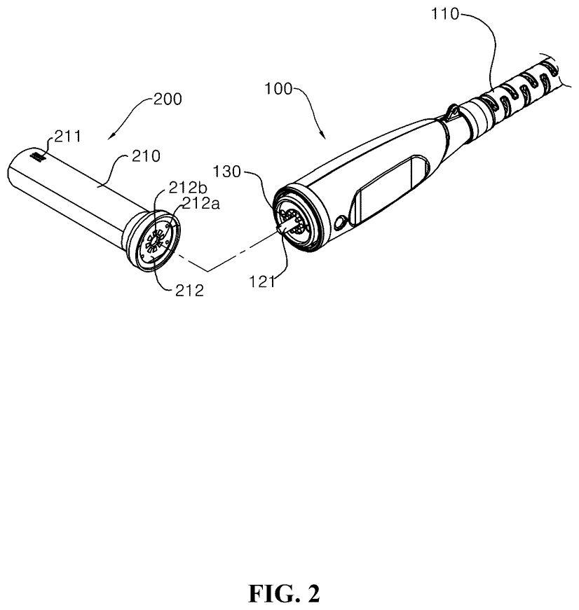

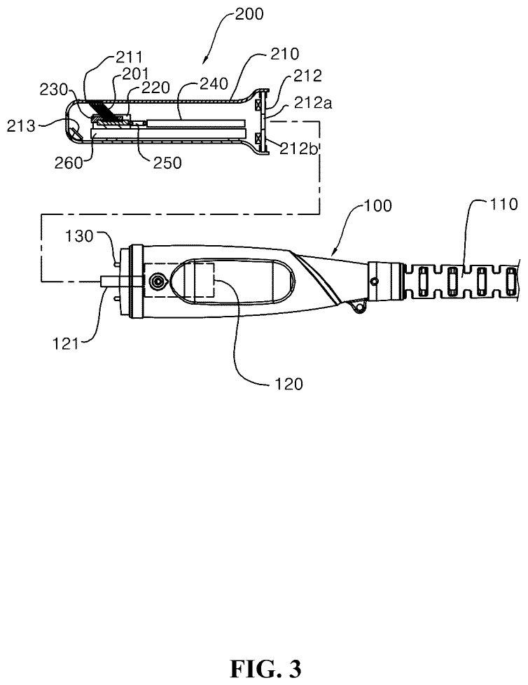

A body-insertable microneedle medical device according to the present invention is formed to be gripped by the hand of an operator, as shown in FIGS. 1 to 3. The body-insertable microneedle medical device includes a handle module 100 having one end connected to a cable 110 configured to receive external power and a driving means 120 installed therein, the handle module being configured such that a moving shaft 121 of the driving means 120 protrudes from a side opposite to the cable 110, and a body-insertable module 200 detachably coupled to the other end of the handle module 100 and configured to receive driving force from the driving means 120 while inserted into the body of a patient such that a plurality of microneedles 201 are exposed to the outside of the body-insertable module 200 to penetrate the body of the patient.

According to the present invention, an operator (doctor) grips the handle module 100 and inserts the body-insertable module 200 into the body of the patient, thereby performing various procedures using the microneedles 201 of the medical device.

A characteristic of the present invention is that, as shown in FIG. 1, the body-insertable module 200 and the handle module 100 are not integrally formed but are independently configured in a detachable manner, thereby forming a structure in which the body-insertable module 200 and the handle module 100 are aligned and connected to each other in a horizontal straight line. Further, this technology allows a portion inserted into the body, that is, the body-insertable module 200, to be discarded after a single use and replaced with a new body-insertable module 200, thereby making it possible to prevent bacterial and viral infections resulting from repeated use.

The handle module 100, as the name suggests, is formed to be gripped by the hand of the operator, and the cable 110 is connected to the rear side of the handle module 100. The cable 110 is used for power supply and communication. The handle module 100 is integrally coupled to t he cable 110, and the driving means 120 is installed inside the handle module 100.

The moving shaft 121 of the driving means 120 is installed on the side opposite to the cable 110. Here, the moving shaft 121 protrudes to the outside of the handle module 100. The protruding moving shaft 121 is connected to the body-insertable module 200 to transmit driving force thereto. Furthermore, since the driving means 120 is implemented as a linear actuator, the moving shaft 121 performs linear motion. Linear actuators are classified into mechanical, electromechanical, hydraulic, and pneumatic types. Preferably, an electromechanical linear actuator using an electric motor such as a stepper motor is applied to the present invention.

Further, the handle module 100 has probe pins 130 formed at portions in contact with the body-insertable module 200 and configured to be electrically connected thereto.

The body-insertable module 200 according to the present invention is a module inserted into the body of a patient and is detachably coupled to one end of the handle module 100. More specifically, the one end corresponds to a direction in which the moving shaft 121 of the handle module 100 and the terminals thereof are exposed. Furthermore, the body-insertable module 200 is provided with a plurality of microneedles 201. Here, the microneedles 201 penetrate the skin tissues of the patient while inserted into the body of the patient, thereby allowing medical procedures to be performed.

The medical procedures using the microneedles 201 may be performed by providing electrical stimulation with microcurrent while the needles are inserted into the patient's body or may be performed by injecting a small amount of medication therethrough. Since such medical procedures using the microneedles 201 are well-known in the art, detailed description thereof will be omitted in the present invention.

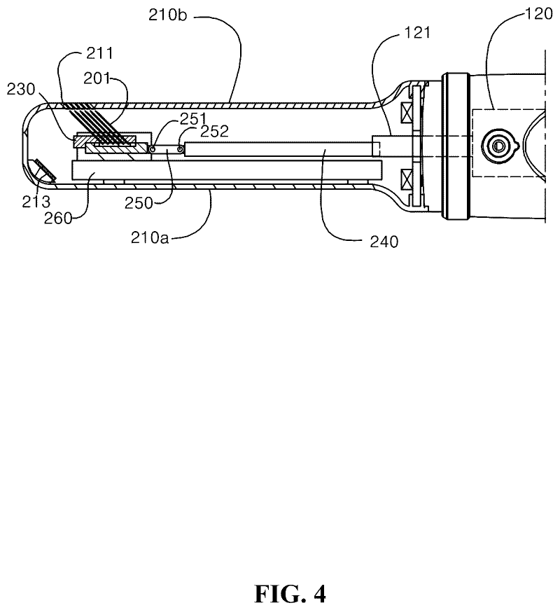

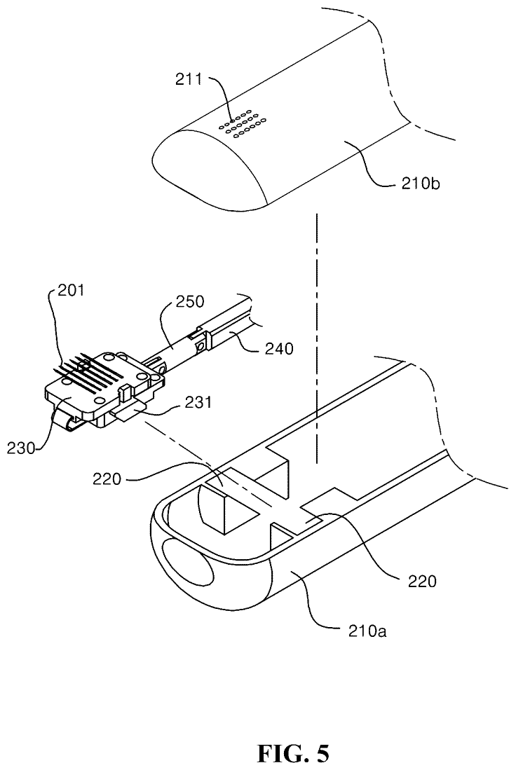

As shown in FIG. 4 or 5, the body-insertable module 200 according to the present invention includes a case 210 having a plurality of needle holes 211 formed through an upper portion thereof, guides 220 integrally formed inside the case 210, a needle block 230 insertably coupled to and guided by the guides 220 and formed to have a plurality of microneedles 201 fixed to an upper portion thereof, and a push bar 240 having one end coupled to the needle block 230 and the other end coupled to the moving shaft 121 to move the needle block 230 by the power of the driving means 120.

The case 210 is formed to have a circular or elliptical horizontal shape to be easily inserted into the body and has an internal space formed therein. Furthermore, a substrate 212 having a through-hole 212a formed therein and a contact terminal 212b formed thereon is installed at a portion of the case 210, which is in contact with the handle module 100, to establish an electrical connection with the body-insertable module 200 and to enable smooth power transmission.

Furthermore, as shown in FIG. 5, the case 210 includes an upper cover 210b and a lower cover 210a for manufacturing convenience. The lower cover 210a is preferably formed to be larger than the upper cover 210b instead of dividing the lower cover 210a and upper cover 210b into halves, as shown in the drawing, so that the guides 220 are formed only on the lower cover 210a and main components are installed thereon. Additionally, a plurality of needle holes 211 are formed in the upper cover 210b. In this manner, when the microneedles 201 move upwards, tip portions of the needles are exposed to the outside through the needle holes 211.

As described above, the guides 220 are integrally formed inside the case 210 and are configured to guide the needle block 230 to move along a specific path. The guides 220 are respectively formed on opposite sides of the needle block 230.

The needle block 230 is configured to have a plurality of microneedles 201 installed thereon. Guide protrusions 231 are formed to protrude from opposite sides of the needle block 230 and are fitted into the respective guides 220.

The needle block 230 is coupled to and guided by the guides 220 in a state of being fitted thereinto and has a plurality of microneedles 201 fixed to an upper portion thereof. The microneedles 201 of the needle block 230 are obliquely fixed in a diagonal direction at a predetermined inclination angle. The guides 220 are also formed to correspond to the inclination angles of the microneedles 201, thereby allowing the needle block 230 to move upwards along the inclination angles. Further, the needle holes 211 through which the microneedles 201 pass are also required to be formed in a diagonal direction.

The reason why the microneedles 201 are configured to be inclined in the diagonal direction is that, in a case in which the surface into which the microneedles 201 are inserted, such as the inside of the vagina, is uneven, inserting the needles in the diagonal direction ensures smoother insertion.

The push bar 240 is configured such that one end thereof is coupled to the needle block 230 and the other end thereof is coupled to the moving shaft 121 to move the needle block 230 by power of the driving means 120. The push bar 240 is formed in a rod shape as shown in the drawing and is coupled to the moving shaft 121.

The push bar 240 and the moving shaft 121 may be coupled to each other in various ways, and the present invention is not limited thereto as long as power can be transmitted without separation during operation. Furthermore, preferably, coupling between the handle module 100 and the body-insertable module 200 is configured such that the handle module 100 and the body-insertable module 200 are separated from each other without excessive difficulty. For example, various configurations such as screw coupling, hole insertion, and engaging only in the direction of movement may be adopted.

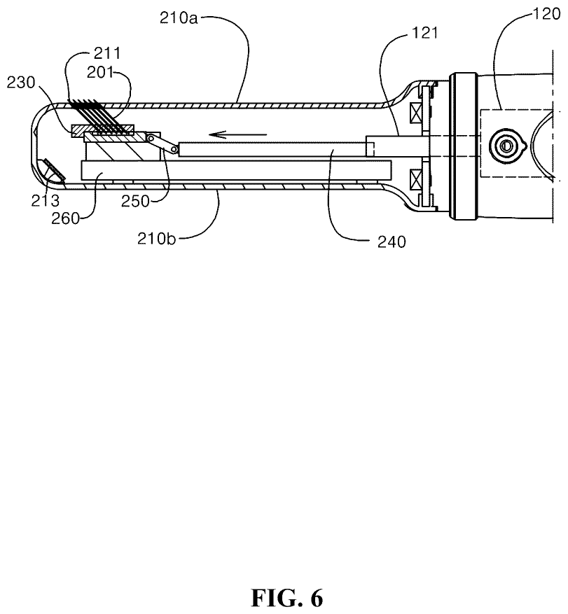

As shown in the drawings, the needle block 230 and the push bar 240 are coupled to each other in such a manner that a link bar 250 having a first rotating part 251 and a second rotating part 252 is coupled between the needle block 230 and the push bar 240 to enable the needle block 230 to move in the diagonal direction. By coupling the link bar 250 including two rotating parts thereto, as shown in FIG. 6, even when the push bar 240 moves horizontally, the needle block 230 may move upwards in the diagonal direction.

As shown in the drawings, the body-insertable module 200 according to the present invention includes the case 210 made of a transparent material, and an imaging means 260 equipped with a camera is further installed below the push bar 240. Accordingly, the inside of the body may be monitored in real time during the insertion process. That is, the imaging means 260 allows the operator to visually inspect the inside of the body and to ensure that the microneedles 201 accurately penetrate a portion requiring treatment. In addition to the camera, an LED may be further installed in the imaging means 260 to provide illumination for enhanced visibility.

Preferably, the imaging means 260 is positioned below the push bar 240 and is formed to have a rod shape. In this case, the camera is installed at the end of the rod. To more accurately identify a treatment portion of the microneedles 201, a reflector 213 may be obliquely installed inside the case 210 in the insertion direction of the microneedles 201.

The images captured in this manner may be viewed through a separate monitoring device (not shown) via the cable 110.

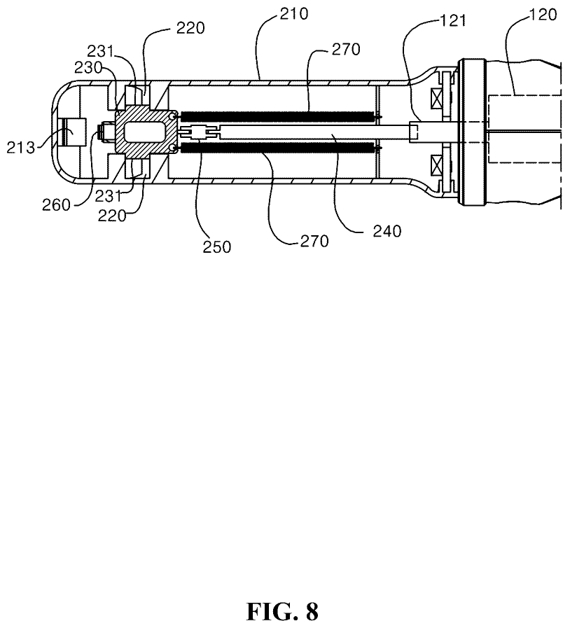

FIG. 8 shows another embodiment of the present invention, which further includes a return spring 270 having one end fixed to a fixing plate provided inside the case 210 and the other end coupled to the needle block 230 to generate pulling force through which the needle block 230 is biased toward the handle module 100. The return spring 270 assists rearward movement of the needle block 230 when the needle block 230 is returned from an advanced state by the push bar 240, particularly when rearward movement of the push bar 240 is not smooth.

That is, in a case in which it is difficult to return the needle block 230 to the original position thereof only by the force of the driving means 120, or in a case in which the moving shaft 121 and the push bar 240 are not properly coupled to each other, the needle block 230 is returned to the original position thereof by the return spring 270.

Although the present invention has been described above with reference to the above-described embodiments, it is to be understood that various modifications are possible within the scope of the technical concept of the present invention.

1. A body-insertable microneedle medical device, comprising:
a handle module formed to be gripped by a hand of an operator, wherein the handle module has one end connected to a cable configured to receive external power, comprises a driving means installed therein, and is configured such that a moving shaft of the driving means protrudes from a side opposite to the cable; and
a body-insertable module detachably coupled to the other end of the handle module, wherein the body-insertable module is configured to receive driving force from the driving means in a state of being inserted into a body of a patient, such that a plurality of microneedles are exposed to an outside of the body-insertable module to penetrate the body of the patient,
wherein the body-insertable module and the handle module are configured independently and connected to each other to form a structure in which the body-insertable module and the handle module are connected to each other in a horizontal straight line,
wherein the body-insertable module comprises:
a case having a plurality of needle holes formed through an upper portion thereof;
guides integrally formed inside the case;
a needle block insertably coupled to and guided by the guides and formed to have the plurality of microneedles fixed to an upper portion thereof; and
a push bar having one end coupled to the needle block and the other end coupled to the moving shaft to move the needle block by power of the driving means,
wherein the case is formed to have a linear horizontal shape with an internal space formed therein and has a substrate installed at a portion in contact with the handle module to establish an electrical connection with the body-insertable module and enable power transmission, wherein the substrate has a through-hole formed therein and a contact terminal formed thereon,
wherein the needle block and the push bar are coupled to each other via a link bar having a first rotating part and a second rotating part to enable the needle block to move in a diagonal direction when the push bar moves horizontally, and
wherein the guides are integrally formed inside the case and are disposed on opposite sides of the needle block, and the needle block has guide protrusions protruding from opposite sides thereof to be coupled to the respective guides by being fitted thereinto,
the body-insertable microneedle medical device further comprising a return spring having one end fixed to a fixing plate provided inside the case and the other end coupled to the needle block to generate pulling force to bias the needle block toward the handle module,
wherein the microneedles of the needle block are obliquely fixed in the diagonal direction at a predetermined angle, and the guides are formed to correspond to inclination angles of the microneedles such that the needle block moves upwards along the inclination angles.

2. The body-insertable microneedle medical device according to claim 1, wherein the case is made of a transparent material, and an imaging means equipped with a camera is further installed below the push bar to monitor an inside of the body in real time during an insertion process.
